(19) United States
(12) Patent Application Publication
(10) Pub. No.: US 2024/0002252 A1
(43) Pub. Date: Jan. 4, 2024

(54) METHODS FOR OPERATING DOMESTIC APPLIANCES INCLUDING MUNICIPAL WATER SUPPLIES

(71) Applicant: Haier US Appliance Solutions, Inc., Wilmington, DE (US)

(72) Inventors: Haitian Hu, Louisville, KY (US); Hairong Li, Louisville, KY (US)

(21) Appl. No.: 17/854,387

(22) Filed: Jun. 30, 2022

(51) Int. Cl. C02F 1/00; G01N 33/18; G08B 21/10

(52) U.S. Cl. CPC C02F 1/003; G01N 33/1826; G08B 21/10

(57) ABSTRACT

A method of operating a domestic appliance includes receiving information from one or more remote sources relating to a quality of water from a municipal water supply to domestic appliance, determining a health advisory has been issued regarding a geographical location of the domestic appliance in response to receiving the information, determining a schedule for performing one or more responsive actions in response to determining that the health advisory has been issued, directing an emission of a first notification, the first notification including one or more sets of instructions, and initiating at least one responsive action of the one or more responsive actions within the domestic appliance after directing the emission of the first notification, the at least one responsive action including restricting an output of a dispenser of the domestic appliance.

### FIELD OF THE INVENTION

The present subject matter relates generally to domestic appliances, and more particularly to method for conditionally operating domestic appliances according to third party information.

### BACKGROUND OF THE INVENTION

Refrigerator appliances generally include one or more cabinets defining chambers for the receipt of food items for storage. Refrigerator appliances may also include features for dispensing ice and/or liquid water. To provide ice and/or liquid water, a dispenser is typically positioned on a door of the appliance. The user positions a container proximate the dispenser, and ice and/or liquid water are deposited into the container depending upon the user's selection. A paddle or other type switch may be provided whereby the user may make a selection. Typically, the liquid water is supplied from a municipal water connection such as a city or local water supply.

Many areas experience issues which may result in contamination to the water supply. For instance, broken water main pipes, industrial accidents, construction, or other activities may lead to health safety advisory actions being issued. Typically, these actions require any incoming water to be properly treated before being consumed to avoid potential illnesses. However, some users of these appliances may be unaware of a majority of advisories issued, which may result in unintended consumption of contaminated water. Current systems may alert users to potential advisories, however further improvements are necessary to effectively enact procedures in the event of an advisory.

Accordingly, a domestic appliance which obviates one or more of the above-mentioned drawbacks would be beneficial. In particular, a method of operating a domestic appliance which initiates one or more determined actions in the event of certain advisories would be useful.

### BRIEF DESCRIPTION OF THE INVENTION

Aspects and advantages of the invention will be set forth in part in the following description, or may be obvious from the description, or may be learned through practice of the invention.

In one exemplary aspect of the present disclosure, a method of operating a domestic appliance is provided. The domestic appliance may include a water supply inlet and a dispenser. The method may include receiving information from one or more remote sources relating to a quality of water from a municipal water supply to the water supply inlet, determining a health advisory has been issued regarding a geographical location of the domestic appliance in response to receiving the information, determining a schedule for performing one or more responsive actions in response to determining that the health advisory has been issued, directing an emission of a first notification, the first notification comprising one or more sets of instructions, and initiating at least one responsive action of the one or more responsive actions within the domestic appliance after directing the emission of the first notification, the at least one responsive action including restricting an output of the dispenser.

In another exemplary aspect of the present disclosure, a domestic appliance is provided. The domestic appliance may include a cabinet forming a receiving space, a water supply inlet provided on the cabinet and through which water is supplied to the receiving space from a municipal water supply, a dispenser provided on the cabinet, the dispenser being fluidly connected with the water supply inlet, and one or more controllers operably connected with the dispenser and the water supply inlet, the one or more controllers including a wireless communication module to allow for a remote connection, the one or more controllers configured to perform an operation. The operation may include receiving information from one or more remote sources relating to a quality of water from the municipal water supply to the water supply inlet, determining a health advisory has been issued at a geographical location of the domestic appliance upon receiving the information, determining a schedule for performing one or more responsive actions in response to determining that the health advisory has been issued, directing an emission of a first notification, the first notification comprising one or more sets of instructions, and initiating at least one responsive action of the one or more responsive actions within the domestic appliance after directing the emission of the first notification, the at least one responsive action including restricting an output of the dispenser.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a front, elevation view of a refrigerator appliance according to exemplary embodiments of the present disclosure.

FIG. 2 provides a front, elevation view a dispensing assembly of the exemplary refrigerator appliance of FIG. 1.

FIG. 3 provides a perspective view of an interior of a door of the exemplary refrigerator appliance of FIG. 1 showing an ice maker.

FIG. 4 provides a schematic illustration of an exemplary refrigerator appliance in communication with a remote user interface device according to one or more embodiments of the present disclosure.

FIG. 5 provides a schematic illustration of an exemplary refrigerator appliance in communication with a remote network according to one or more embodiments of the present disclosure.

FIG. 6 provides a flow chart illustrating a method of operating a refrigerator appliance.

Repeat use of reference characters in the present specification and drawings is intended to represent the same or analogous features or elements of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “first,” “second,” and “third” may be used interchangeably to distinguish one component from another and are not intended to signify location or importance of the individual components. The terms “includes” and “including” are intended to be inclusive in a manner similar to the term “comprising.” Similarly, the term “or” is generally intended to be inclusive (i.e., “A or B” is intended to mean “A or B or both”). In addition, here and throughout the specification and claims, range limitations may be combined and/or interchanged. Such ranges are identified and include all the sub-ranges contained therein unless context or language indicates otherwise. For example, all ranges disclosed herein are inclusive of the endpoints, and the endpoints are independently combinable with each other. The singular forms “a,” “an,” and “the” include plural references unless the context clearly dictates otherwise.

Approximating language, as used herein throughout the specification and claims, may be applied to modify any quantitative representation that could permissibly vary without resulting in a change in the basic function to which it is related. Accordingly, a value modified by a term or terms, such as “generally,” “about,” “approximately,” and “substantially,” are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a 10 percent margin, i.e., including values within ten percent greater or less than the stated value. In this regard, for example, when used in the context of an angle or direction, such terms include within ten degrees greater or less than the stated angle or direction, e.g., “generally vertical” includes forming an angle of up to ten degrees in any direction, e.g., clockwise or counterclockwise, with the vertical direction V.

The word “exemplary” is used herein to mean “serving as an example, instance, or illustration.” In addition, references to “an embodiment” or “one embodiment” does not necessarily refer to the same embodiment, although it may. Any implementation described herein as “exemplary” or “an embodiment” is not necessarily to be construed as preferred or advantageous over other implementations. Moreover, each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

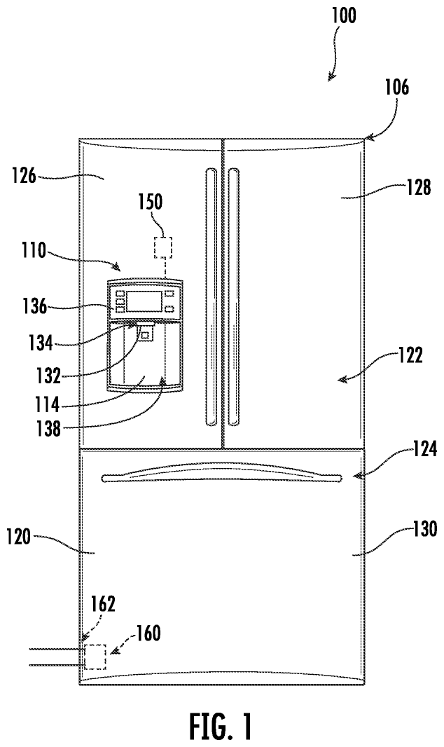

Referring now to the figures, FIG. 1 depicts a front view of an example embodiment of a refrigerator appliance 100. Refrigerator appliance 100 includes a cabinet or housing 120 defining an upper fresh food chamber 122 and a lower freezer chamber 124 arranged below the fresh food chamber 122. As such, refrigerator appliance 100 is generally referred to as a bottom-mount refrigerator appliance. In the exemplary embodiment, housing 120 also defines a mechanical compartment (not shown) for receipt of a sealed cooling system. Using the teachings disclosed herein, one of skill in the art will understand that the present invention may be used with other types of refrigerator appliances (e.g., side-by-sides or top-mounts). Consequently, the description set forth herein is for illustrative purposes only and is not intended to limit the invention to any particular style of refrigerator appliance or arrangement of chilled chambers.

Refrigerator doors 126, 128 are rotatably hinged to an edge of housing 120 for accessing fresh food compartment 122. A freezer door 130 is arranged below refrigerator doors 126, 128 for accessing freezer chamber 124. In the exemplary embodiment, freezer door 130 is coupled to a freezer drawer (not shown) slidably mounted within freezer chamber 124.

Refrigerator appliance 100 includes a dispensing assembly 110 for dispensing liquid water and ice. Dispensing assembly 110 includes a dispenser 114 positioned on an exterior portion of refrigerator appliance 100. Dispenser 114 includes a discharging outlet 134 for accessing ice and liquid water. Dispensing assembly 110 further includes a sensor 112 positioned on discharging outlet 134. As will be described in more detail below, sensor 112 may be configured to detect a presence of a container positioned within dispensing assembly 110, and to detect the top lip of the container. A user interface panel 136 is provided for controlling the mode of operation. For example, user interface panel 136 includes a water dispensing button (not labeled) and an ice-dispensing button (not labeled) for selecting a desired mode of operation such as crushed, non-crushed ice, or liquid water, etc.

Discharging outlet 134 is an external part of dispenser 114, and is mounted in a dispensing recess or recessed portion 138 defined in an outside surface of refrigerator door 126. Recessed portion 138 is positioned at a predetermined elevation convenient for a user to access ice or liquid water and enabling the user to access ice or liquid water without the need to bend-over and without the need to access freezer chamber 124. In the exemplary embodiment, recessed portion 138 is positioned at a level that approximates the chest level of a user.

Refrigerator appliance 100 may include a valve 160. Valve 160 may selectively allow a flow of water (e.g., from a municipal water supply) into refrigerator appliance 100. Thus, valve 160 may be provided on a water supply inlet 162 of refrigerator appliance 100. Valve 160 may be an electromechanical valve, for instance. However, it should be understood that the valve may be any suitable valve, such as a mechanical valve (with an attached servo motor), a solenoid valve, a motor valve, or the like. Valve 160 may selectively open or close according to a signal input from a controller (described below).

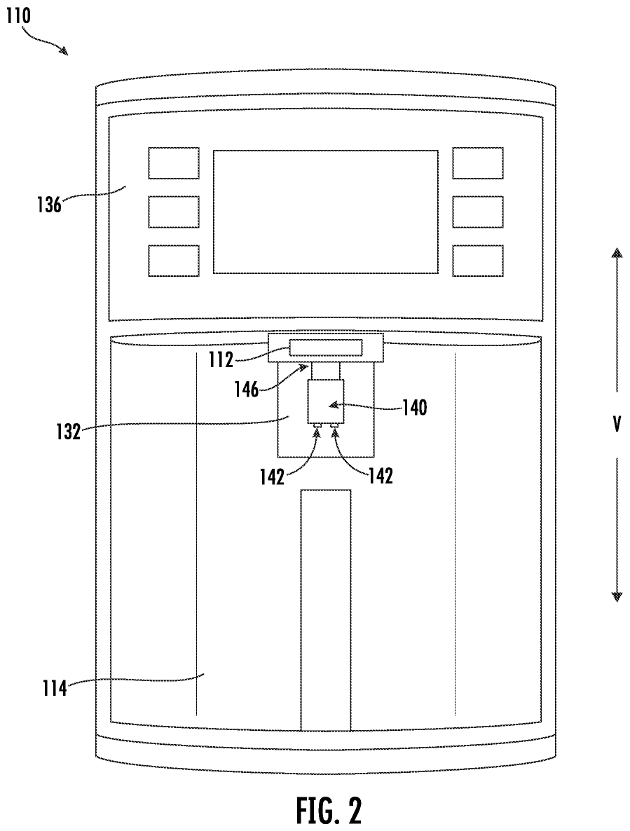

FIG. 2 provides a close-up front view of the dispenser 114 of dispensing assembly 110. An exemplary nozzle 140 is positioned adjacent to an activation member 132. For one example, activation member 132 is an activation paddle configured to be manipulated (e.g., by a receptacle or vessel to collect liquid from nozzle 140). Nozzle 140 includes a plurality of fluid outlets 142 through which liquid water may flow into a container placed into the recess 138 of dispensing assembly 110 by a user of appliance 100. Dispensing assembly 110 may further include a sensor, such as sensor 112. Sensor 112 may be positioned above nozzle 140 within dispenser 114. In particular, sensor 112 may be positioned within an upper portion of dispenser 114 such that one or more signals generated by sensor 112 are transmitted parallel to the liquid water stream. In this manner, sensor 112 may be positioned vertically above a container placed in dispenser 114.

In example embodiments, sensor 112 may be an ultrasonic transducer configured to periodically transmit and receive high frequency sound waves, and to convert the received sound waves into electrical data. In particular, sensor 112 may be configured to generate and transmit a sound wave, and to receive one or more echoed sound waves. Sensor 112 may further be configured to determine a time interval between transmitting the sound wave and receiving the one or more echoes. It will be appreciated that various other sensors and/or sensor configurations may be used, such as for instance, a sensor configuration including a separate and distinct transmitter and receiver. For instance, sensor 112 may be any suitable sensor, such as an infrared sensor, an optical sensor, a laser sensor, a capacitive sensor, or an inductive sensor, e.g., directed towards dispenser recess 138 of refrigerator appliance 100 for assisting operation of dispenser 114.

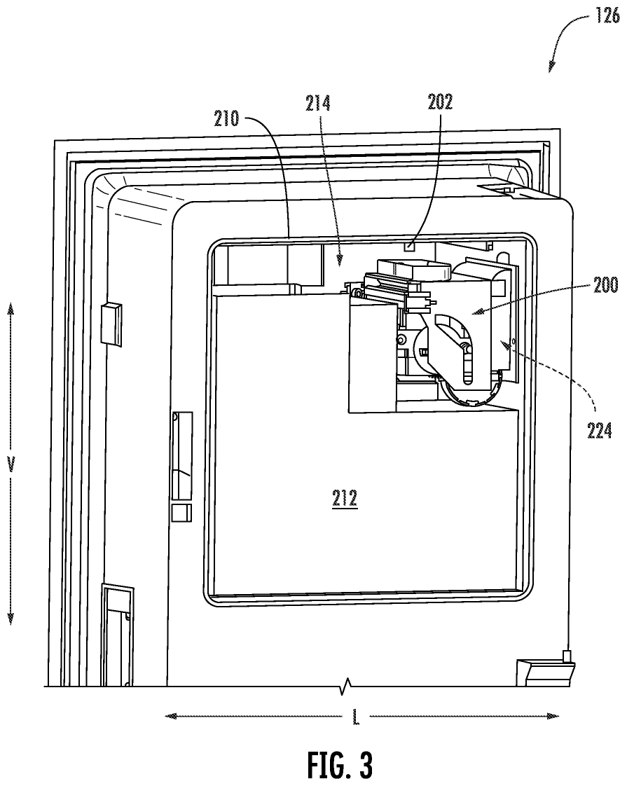

Referring now to FIG. 3, an ice making assembly 200 may be provided within refrigerator appliance 100. As illustrated, ice making assembly 200 may be mounted on an icebox 210 within an ice making chamber 214 and is configured for receiving a flow of water from water supply input or spout 202 (see, e.g., FIG. 1). In this manner, ice making assembly 200 may be generally configured for freezing the water to form ice cubes 204 which may be stored in a storage bin 212 and dispensed through discharging outlet 146 by dispensing assembly 110. However, it should be appreciated that ice making assembly 200 is described herein only for the purpose of explaining aspects of the present subject matter. Variations and modifications may be made to ice making assembly 200 while remaining within the scope of the present subject matter. For example, ice making assembly 200 could instead be positioned within freezer chamber 124 of refrigerator appliance 100 and may have any other suitable configuration.

Refrigerator appliance 100 further includes a controller 150. Operation of the refrigerator appliance 100 is regulated by controller 150 that is operatively coupled to user input panel 136. In one exemplary embodiment, user input panel 136 may represent a general purpose I/O (“GPIO”) device or functional block. In another exemplary embodiment, user input panel 136 may include input components, such as one or more of a variety of electrical, mechanical or electro-mechanical input devices including rotary dials, push buttons, and touch pads. User input panel 136 may be in communication with controller 150 via one or more signal lines or shared communication busses.

User input panel (or user input) 136 provides selections for user manipulation of the operation of refrigerator appliance 100. In response to user manipulation of the user input panel 136, controller 150 operates various components of refrigerator appliance 100. For example, controller 150 is operatively coupled or in communication with actuator 132, first ultrasonic sensor 152, and second ultrasonic sensor 154, such that controller 150 can operate such components. In particular, controller 150 is in communication with first and second ultrasonic sensors 152 and 154 and may receive signals from such components. Controller 150 can receive such signals in order to detect or locate a container within dispenser recess 138 as discussed above.

Controller 150 includes memory and one or more processing devices such as microprocessors, CPUs or the like, such as general or special purpose microprocessors operable to execute programming instructions or micro-control code associated with operation of refrigerator appliance 100. The memory can represent random access memory such as DRAM, or read only memory such as ROM or FLASH. The processor executes programming instructions stored in the memory. The memory can be a separate component from the processor or can be included onboard within the processor. Alternatively, controller 150 may be constructed without using a microprocessor, e.g., using a combination of discrete analog and/or digital logic circuitry (such as switches, amplifiers, integrators, comparators, flip-flops, AND gates, and the like) to perform control functionality instead of relying upon software.

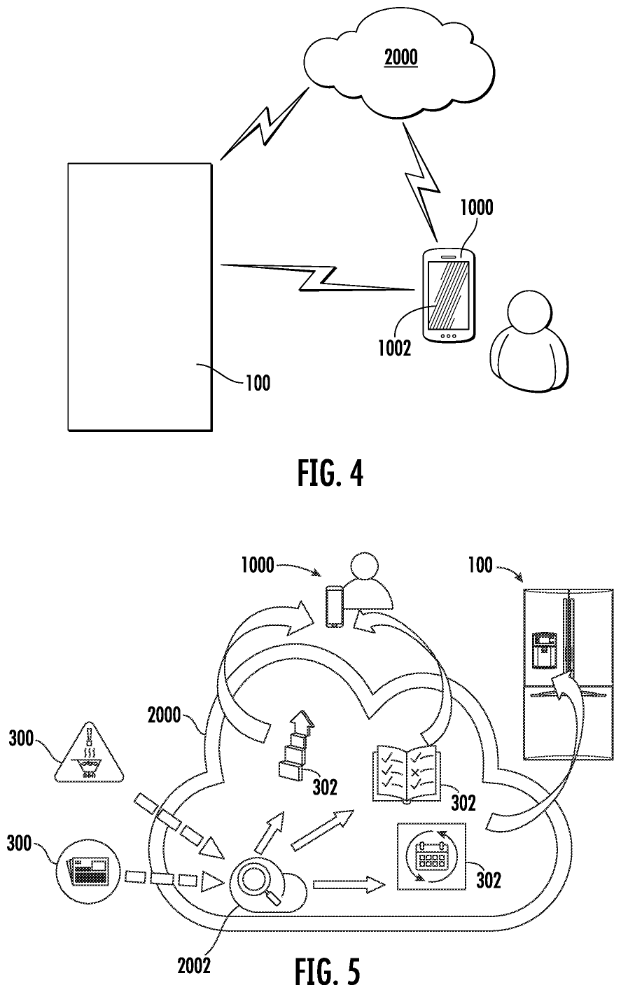

FIG. 4 schematically illustrates the refrigerator appliance 100 communicating with a remote user interface device 1000. Also shown (but not numbered) in FIG. 4 is a user such as may interact with the remote user interface device 1000, e.g., via a user interface 1002 of the remote user interface such as a touchscreen in the illustrated embodiment. For example, the remote user interface device 1000 may be a device such as a cell phone, smart phone, smart assistant, or any similar device in operative communication with the controller 150 via a wireless connection. As shown in FIG. 4, the refrigerator appliance 100, and in particular, controller 150 thereof, may be configured to communicate with a separate device external to the appliance 100, such as a communications device or other remote user interface device 1000. The remote user interface device 1000 may be a laptop computer, smartphone, tablet, personal computer, wearable device, smart home system (such as a smart assistant speaker), or various other suitable devices. The refrigerator appliance 100 may include a network communication module, e.g., a wireless communication module, for communicating with the remote user interface device 1000. In various embodiments, a network communication module may include a network interface such that the controller 150 of the refrigerator appliance 100 can connect to and communicate over one or more networks with one or more network nodes. A network communication module may also include one or more transmitting, receiving, or transceiving components for transmitting/receiving communications with other devices communicatively coupled with refrigerator appliance 100. The network communication module may be in communication with, e.g., coupled or connected to, the controller 150 to transmit signals to and receive signals from the controller 150.

As schematically illustrated in FIG. 4, the refrigerator appliance 100 may be configured to communicate with the remote user interface device 1000 either directly or through a network 2000 (e.g., a smart home network). Thus, in various embodiments, the refrigerator appliance 100 and the remote user interface 1000 may be configured to communicate wirelessly with each other or with the network 2000. The network 2000 may be or include various possible communication connections and interfaces, e.g., such as Zigbee, BLUETOOTH®, WI-FI®, or any other suitable communication connection. The remote user interface device 1000 may include a memory for storing and retrieving programming instructions. For example, the remote user interface device 1000 may be a smartphone operable to store and run applications, also known as “apps,” and may include a remote user interface provided as a smartphone app. Additionally or alternatively, multiple remote user interface devices 1000 may be connected with refrigerator appliance 100. For instance, one or more smart phones, one or more smart assistant devices (smart speakers), or the like may be simultaneously connected with refrigerator appliance 100 (e.g., through network 2000).

FIG. 5 provides a graphical representation of a system 170 including appliance 100 and user interface device 1000. For instance, the system 170 may include network 2000. One or more remote sources 300 (e.g., news sources, social media networks, search engines, etc.) may be connected to network 2000. Thus, information from the one or more remote sources 300 may be selectively provided to appliance 100, user interface device 1000, or additional remote devices. The network may include a remote server 2002. The remote server 2002 may collect, compile, organize, analyze, distribute, etc., the information collected from remote sources 300. Additionally or alternatively, remote server 2002 may develop or otherwise formulate informational data files 302 (e.g., guides, tips, instructions, etc.). The informational data files may be selectively distributed to appliance 100, user interface device 1000, or the like, as will be explained in further detail below.

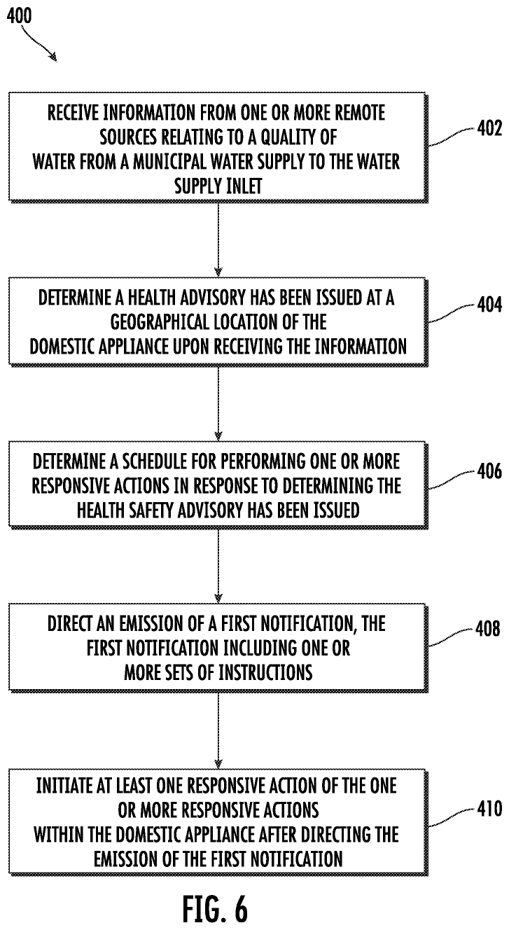

FIG. 6 depicts a flow diagram of an example method 400 of operating a domestic (e.g., refrigerating) appliance according to a specific embodiment of the present disclosure. The controller 150 of appliance 100 may be configured or programmed to implement method 400. In addition, FIG. 6 depicts steps performed in a particular order for purposes of illustration and discussion. Those of ordinary skill in the art, using the disclosures provided herein, will understand that the steps of any of the methods disclosed herein may be modified, adapted, expanded, omitted, and/or rearranged in various ways without deviating from the scope of the present disclosure. Additionally or alternatively, a separate dedicated controller may be configured to perform method 400. Further still, method 400 may be applied to any suitable domestic appliance configured to supply water (e.g., liquid water, ice, etc.) to one or more users (e.g., a stand-alone ice dispenser, a stand-alone beverage dispenser, a water cooler, etc.).

At step 402, method 400 may include receiving information from one or more remote sources relating to a quality of water from a municipal water supply to the water supply inlet. In detail, the appliance (e.g., refrigerator appliance 100) may, through a wireless connection to a wireless network, receive at least one informational notification regarding a potential or impending water advisory, such as a boil water advisory. The remote sources may include local or regional news channels or outlets, utilities agencies, health boards, social media networks (e.g., Twitter®, Facebook®, etc.), community message boards, or the like. For instance, the controller of the appliance may periodically scan the one or more remote sources for information relating to water quality issues. Additionally or alternatively, the appliance may include one or more algorithms configured to retrieve or import water-quality specific alerts from the one or more sources.

The one or more remote sources may include a separate remotely connected appliance. For instance, a plurality of appliances may be interconnected via a cloud or wireless network, for example through a remote server. The plurality of appliances may be provided remotely to each other (e.g., in different households or physical locations). Additionally or alternatively, each of the plurality of appliances may belong to or be registered to different unique users. Accordingly, a first remote appliance may receive an input regarding a water quality issue directly from a user. For example, a first user associated with the first remote appliance independently discovers the water quality issue and inputs the discovered information to the first remote appliance. The first remote appliance may then upload the water quality issue to the remotely connected server or cloud. The remotely connected server or cloud may then distribute or communicate the water quality issue to a second remote appliance.

At step 404, method 400 may include determining a health advisory has been issued regarding a geographical location of the domestic appliance in response to receiving the information. In detail, the appliance may analyze the information (e.g., via the one or more controllers). The appliance may then determine that a particular health advisory has been issued. For instance, the appliance determines that a boil water advisory has been instituted or scheduled within a predetermined time. The appliance may determine that the health advisory will affect a geographical location in which the appliance is located.

In detail, the appliance may include location data for itself and the health advisory. The location data of the appliance itself may be procured via an onboard global positioning system (GPS) device. The location data of the health advisory may be gleaned from an analysis of the information received at step 402. For instance, the information may include one or more of a city, a county, a zip code, an area code, a neighborhood, a water supply network, or the like in which the health advisory is to take effect. The appliance may then overlap its own geographical location with the information received to determine if the water supplied thereto will be affected.

At step 406, method 400 may include determining a schedule for performing one or more responsive actions in response to determining the health safety advisory has been issued. In detail, the appliance may determine, ascertain, or otherwise formulate a timeframe for the health advisory. The schedule may include a start time of the health advisory. For instance, upon analyzing the information received at step 402, the appliance may determine that the health advisory (e.g., a boil water advisory) is set or predicted to initiate at a certain time of day on a certain date (e.g., 12:00 AM 2 days from receiving the information). Thus, a schedule may be developed for implementing one or more responsive actions around the initiation of the health advisory.

The one or more responsive actions may include operational changes to the appliance. According to one example, the appliance is a refrigerator appliance including a dispenser (e.g., a liquid dispenser selectively dispensing liquid water to a user on demand). As described above, the dispenser may be fluidly connected with a municipal water supply, such as a city water supply. Upon determining that the health safety advisory has been established, a responsive action may include restricting an operation of the dispenser. In detail, the refrigerator appliance may disable the dispenser such that water is unable to be dispensed therefrom for a predetermined amount of time. The refrigerator appliance (e.g., a controller) may break or disrupt electrical signals between the controller and the dispenser as the responsive action. Accordingly, a user input (e.g., manipulation of an activation paddle, a press of a button, a voice command, etc.) may be ignored by the dispenser, and water is not dispensed despite receiving the user input.

According to another embodiment, the refrigerator appliance includes an automatic ice maker. The automatic ice maker may selectively receive water from the municipal water supply into one or more ice molds to form ice. Thus, a responsive action may include restricting an operation of the ice maker. For instance, the refrigerator appliance (e.g., controller) may disable the ice maker such that water is not supplied thereto for a predetermined amount of time. The appliance (e.g., controller) may break an electrical contact between the controller and the ice maker such that any inputs (e.g., automatic or on demand inputs to form ice) are ignored by the ice maker, and water is not supplied to the ice maker despite receiving inputs.

The automatic ice maker may include one or more features for determining a level of ice within an ice storage container or ice bucket storing the formed ice. For instance, the automatic ice maker may include one or more of a camera assembly, a contact switch, a temperature sensor, a thermistor, a level sensor, or the like, capable of determining the level of ice within the ice storage container. A responsive action may include determining a level of ice within the ice storage container. If the appliance determines that the level of ice within the ice storage container is below a predetermined level (e.g., less than full, half full, one quarter full), the responsive action includes instructing the ice maker to perform an ice making operation prior to deactivating the ice maker. The instruction to perform the ice making operation may be conditioned on determining that the health safety advisory is not yet in effect.

In determining the schedule for performing the one or more responsive actions, the method 400 may include determining the start time by which the health safety advisory will be in effect. For instance, the health safety advisory may be set to initiate in the future. Accordingly, the appliance may determine a length or amount of time until the initiation of the health safety advisory. The appliance may then compare the length of time until the initiation of the health safety advisory with a length or amount of time required to perform an ice making operation. In one example, the appliance compares the length of time until the initiation of the health safety advisory with a length or amount of time required to fill the ice storage container with ice formed from the ice maker. When the amount of time required to perform the ice making operation is less than the length of time until the initiation of the health safety advisory, the responsive action includes instructing the ice maker to perform the ice making operation.

According to still another embodiment, the appliance is a stand-alone liquid dispenser connected to the municipal water supply. Similar to the dispenser in the refrigerator appliance, the controller may disable the dispenser such that water from the municipal water supply is not dispensed from the dispenser despite receiving inputs. According to still another embodiment, the appliance is a stand-alone ice maker forming ice from the municipal water source. Similar to the ice maker in the refrigerator appliance, the controller may disable the ice maker such that water from the municipal water supply is not provided or supplied to the ice maker (e.g., the ice mold or ice molds) despite receiving inputs or commands.

The appliance may include a water supply inlet connected to the municipal water supply. In detail, a valve may be provided at the water supply inlet to selectively allow water into the appliance. The valve may be an electromechanical valve, for instance. However, it should be understood that the valve may be any suitable valve, such as a mechanical valve (with an attached servo motor), a solenoid valve, a motor valve, or the like. The valve may be operably connected with the controller to selectively open and close (e.g., according to a signal from the controller). Accordingly, the valve may selectively allow or restrict water (e.g., from the municipal water supply) from entering the appliance.

The one or more responsive actions may thus include closing the valve such that water is not supplied to the appliance. For instance, upon determining that the health safety advisory has been issued, the appliance (e.g., controller) may instruct the valve to close. The instruction for the valve to close may be initiated according to the determined schedule, for instance at a start time of the determined schedule. According to some embodiments, a combination of the potential responsive actions are implemented together (e.g., closing the valve and disabling the dispenser). It should be understood that any suitable combination of responsive actions may be implemented together according to the determined schedule.

In determining the schedule for performing the one or more responsive actions, the method 400 may consider an estimated or set ending time for the health safety advisory. For instance, in receiving the information regarding the health safety advisory, the appliance may determine that an end time of the health safety advisory has been noted by the one or more remote sources. In one example, the appliance determines that the health safety advisory will last for 24 hours. Accordingly, the method may incorporate additional or alternative actions throughout the determined schedule of performing the responsive actions, as will be described in further detail below.

At step 408, method 400 may include directing an emission of a first notification. The first notification may include one or more sets of instructions. In detail, upon receiving the information relating to the quality of water and the health safety advisory, the method 400 may include instructing a notification to be sent to a mobile device (e.g., a mobile phone, smart phone, tablet, personal computer, etc.). The notification may be sent from the appliance itself to the mobile device via a network connection. Additionally or alternatively, the notification may be sent from a remote server or cloud.

The notification may be a push notification transmitted to the mobile device. For instance, the notification may appear as an alert on a user's mobile device (e.g., as a pop-up notification on a smart phone). According to some embodiments, the notification is provided to the mobile device via a mobile application (app) installed on the mobile device. The first notification may be a text or graphic notification. For instance, a textual notification may be presented including the alert of the health safety advisory, a timing for performing the determined schedule, a list of potential responsive actions, an affected area, an estimated length of time of the health safety advisory, or the like. Additionally or alternatively, a graphical representation of the health safety advisory may be included in the notification. For example, a map of the affected area, pictures or graphic representations of appliances, or the like are included in the notification. A combination of text and graphics may be included in the notification.

The first notification may include one or more sets of instructions. In detail, the first notification may include instructions for manually performing at least one of the one or more responsive actions. For one example, the first notification includes a prompt to initiate at least one of the one or more responsive actions immediately. In detail, the first notification may present the user with a selectable option to disable the dispenser, disable the ice maker, close the valve, or the like. The instructions may further include tips or steps to take to ensure any water to be consumed is safely disinfected (e.g., boiling procedures, filtering procedures, temperatures for consumption or use, etc.). The instructions may include one or more to-do lists pertaining to the domestic or consumer appliances provided within the affected area of the health safety advisory. The first notification may include a plurality of the above-mentioned sets of instructions.

At step 410, method 400 may include initiating at least one responsive action of the one or more responsive actions within the domestic appliance after directing the emission of the first notification. As mentioned above, one or more of the responsive actions may be initiated or performed according to the determined schedule. Upon providing the first notification to the user, the appliance may then initiate the one or more responsive actions. For example, after providing the first notification, the appliance disables the dispenser and disables the ice maker. The initiation of the one or more responsive actions may be performed according to the determined schedule. For instance, when the health safety advisory is set to begin 10 hours after notification, the one or more responsive actions may be initiated a predetermined amount of time before the beginning of the health safety advisory (e.g., ten minutes before, thirty minutes before, etc.).

Method 400 may further include determining that the health safety advisory has been lifted or removed. In detail, the appliance may receive additional information from the one or more remote sources indicating that the health safety advisory is no longer in effect. The additional information may indicate that the health safety advisory has particularly been lifted from the geographical location of the appliance. Additionally or alternatively, the appliance may determine that the health safety advisory has been lifted from the initial information (e.g., if a scheduled or predicted end time of the advisory was included in the initial alert).

Accordingly, method 400 may include initiating a post-advisory action in response to determining that the health safety advisory has been lifted or removed. In detail, the post-advisory action may include performing additional responsive actions, The additional responsive actions may include undoing the previously performed responsive actions. For instance, if one of the responsive actions included disabling the dispenser, the post-advisory action includes enabling the dispenser. Similarly, if one of the responsive actions included disabling the automatic ice maker, the post-advisory action includes enabling the automatic ice maker. Accordingly, the post-advisory action may effectively reset the appliance to previous operational settings (e.g., before the health safety advisory).

At least one of the post-advisory actions may include directing an emission of a second notification. The second notification may include one or more sets of reactivation instructions for the appliance. For instance, the second notification may include instructions for enabling the dispenser, enabling the ice maker, opening the valve, flushing currently stagnant water from water supply pipes to the appliance or appliances, changing filters, and the like. According to some embodiments, the post-advisory actions are presented to the user as an augmented reality guide. In detail, virtual instructions may be provided to the user outlining and demonstrating the required steps to perform the post-advisory actions.

The augmented reality instructions may include generating an appliance avatar. The appliance avatar may be displayed on the remote mobile device. For instance, on a display screen of the mobile device, an avatar of the appliance model may be displayed so as to be identical to the physical appliance. The user may initiate a capture of the appliance via a camera assembly of the mobile device. The appliance avatar may provide a controller or computer-generated model, such as a virtual three-dimensional model of the appliance avatar. Advantageously, the user may easily identify certain required elements of the appliance to be manipulated according to the second notification.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A method of operating a domestic appliance, the domestic appliance comprising a water supply inlet and a dispenser, the method comprising:
receiving information from one or more remote sources relating to a quality of water from a municipal water supply to the water supply inlet;
determining a health advisory has been issued regarding a geographical location of the domestic appliance in response to receiving the information;
determining a schedule for performing one or more responsive actions in response to determining that the health advisory has been issued;
directing an emission of a first notification, the first notification comprising one or more sets of instructions; and
initiating at least one responsive action of the one or more responsive actions within the domestic appliance after directing the emission of the first notification, the at least one responsive action comprising restricting an output of the dispenser.

2. The method of claim 1, wherein the domestic appliance further comprises a dispenser to selectively dispense water, and wherein the one or more responsive actions comprises disabling the dispenser such that water is unable to be dispensed therefrom for a predetermined amount of time.

3. The method of claim 1, wherein the domestic appliance further comprises an ice maker to form ice from water supplied from the water supply inlet, and wherein the one or more responsive actions comprises disabling the ice maker such that water is not supplied thereto for a predetermined amount of time.

4. The method of claim 3, further comprising:
determining a start time for the schedule for performing the one or more responsive actions, the amount of time until the start time being greater than or equal to an amount of time required to fill an ice storage container with ice formed from the ice maker; and
initiating an ice making operation upon determining that the amount of time until the start time is greater than or equal to the amount of time required to fill the ice storage container.

5. The method of claim 1, wherein the domestic appliance further comprises a valve provided at the water supply inlet to selectively allow water from the municipal water supply into the domestic appliance, and wherein the one or more responsive actions comprises closing the valve such that water is not supplied to the domestic appliance.

6. The method of claim 1, wherein the first notification is a text or graphic notification transmitted to a personal remote device.

7. The method of claim 6, wherein the one or more sets of instructions comprises a prompt to initiate at least one of the one or more responsive actions immediately.

8. The method of claim 1, further comprising:
determining that the health advisory has been removed from the geographical location of the domestic appliance; and
initiating a post-advisory action in response to determining that the health advisory has been removed.

9. The method of claim 8, wherein initiating the post-advisory action comprises:
directing an emission of a second notification, the second notification comprising one or more sets of reactivation instructions for the domestic appliance.

10. The method of claim 9, wherein the one or more sets of reactivation instructions are provided as an augmented reality guide comprising virtual instructions on a mobile device.

11. A domestic appliance comprising:
a cabinet forming a receiving space;
a water supply inlet provided on the cabinet and through which water is supplied to the receiving space from a municipal water supply;
a dispenser provided on the cabinet, the dispenser being fluidly connected with the water supply inlet; and
one or more controllers operably connected with the dispenser and the water supply inlet, the one or more controllers comprising a wireless communication module to allow for a remote connection, the one or more controllers configured to perform an operation, the operation comprising
receiving information from one or more remote sources relating to a quality of water from the municipal water supply to the water supply inlet;
determining a health advisory has been issued at a geographical location of the domestic appliance upon receiving the information;
determining a schedule for performing one or more responsive actions in response to determining that the health advisory has been issued;
directing an emission of a first notification, the first notification comprising one or more sets of instructions; and
initiating at least one responsive action of the one or more responsive actions within the domestic appliance after directing the emission of the first notification, the at least one responsive action comprising restricting an output of the dispenser.

12. The domestic appliance of claim 11, wherein the one or more responsive actions comprises disabling the dispenser such that water is unable to be dispensed therefrom for a predetermined amount of time.

13. The domestic appliance of claim 11, further comprising an ice maker provided within the receiving space to form ice from water supplied from the water supply inlet, and wherein the one or more responsive actions comprises disabling the ice maker such that water is not supplied thereto for a predetermined amount of time.

14. The domestic appliance of claim 13, wherein the operation further comprises:
determining a start time for the schedule for performing the one or more responsive actions, wherein an amount of time until the start time is greater than or equal to an amount of time required to fill an ice storage container with ice formed from the ice maker; and
initiating an ice making operation upon determining that the amount of time until the start time is greater than or equal to the amount of time required to fill the ice storage container.

15. The domestic appliance of claim 11, further comprising a valve provided at the water supply inlet to selectively allow water from the municipal water supply into the domestic appliance, and wherein the one or more responsive actions comprises closing the valve such that water is not supplied to the domestic appliance.

16. The domestic appliance of claim 11, wherein the first notification is a text or graphic notification transmitted to a personal remote device.

17. The domestic appliance of claim 16, wherein the one or more sets of instructions comprises a prompt to initiate at least one of the one or more responsive actions immediately.

18. The domestic appliance of claim 11, wherein the operation further comprises:
determining that the health advisory has been removed from the geographical location of the domestic appliance; and
initiating a post-advisory action in response to determining that the health advisory has been removed.

19. The domestic appliance of claim 18, wherein initiating the post-advisory action comprises:
directing an emission of a second notification, the second notification comprising one or more sets of reactivation instructions for the domestic appliance.

20. The domestic appliance of claim 19, wherein the one or more sets of reactivation instructions are provided as an augmented reality guide comprising virtual instructions on a mobile device.
